(19) United States
(12) Patent Application Publication
(10) Pub. No.: US 2026/0076323 A1
(43) Pub. Date: Mar. 19, 2026

(54) SYSTEM AND METHOD FOR DISTRIBUTING A GROWTH MEDIA TO PLANTS

(71) Applicant: Lowes TC Pty Ltd, New South Wales (AU)

(72) Inventor: Greg LOWE, New South Wales (AU)

(73) Assignee: Lowes TC Pty Ltd, New South Wales (AU)

(21) Appl. No.: 18/998,413

(22) PCT Filed: Jul. 27, 2023
(86) PCT No.: PCT/AU2023/050696

§ 371 Date: Jan. 24, 2025

(30) Foreign Application Priority Data
Jul. 27, 2022 (AU) 2022902102

(51) Int. Cl. A01H 4/00

(52) U.S. Cl. CPC A01H 4/001

(57) ABSTRACT

The invention relates to a system for distributing a growth media to plants. The system comprises a container for receiving the plants, a first conduit for fluidly connecting the container to a reservoir having the growth media, and a suction device fluidly connected to the container. The suction device is operable to draw air from the container so as to cause the growth media to flow from the reservoir into the container and at least partially immerse the plants.

### FIELD OF THE INVENTION

The invention relates to a system and method for growing plants and in particular to a system and method for distributing a growth media to plants in a container for propagation. The invention has been developed primarily for use in growing plants under plant tissue culture and will be described predominantly in this context. However, it will be appreciated that the invention is not limited to this particular field of use, being potentially applicable in a wide variety of applications including sterile and non-sterile applications, particularly applications based on greenhouse and outdoor environments.

### BACKGROUND OF THE INVENTION

The following discussion of the prior art is intended to present the invention in an appropriate technical context and allow its advantages to be properly appreciated. Unless clearly indicated to the contrary, however, reference to any prior art in this specification should not be construed as an express or implied admission that such art is widely known or forms part of common general knowledge in the field.

Commercial Plant Tissue Culture (PTC) is the clonal micro propagation of plants for the horticultural industry including ornamentals for home and landscape, cut flowers, revegetation, horticultural food crops, pharmaceutical crops and forestry. Historically, PTC has been an expensive way to propagate plants compared to seed and unrooted cutting production (URC) methods, but PTC has found a niche to produce difficult to propagate plants and for plants that must be supplied in a high health status. But for its cost, PTC has the advantages of producing plants with high health, non-seasonality, increased branching and overall early growth vigour.

PTC is traditionally done in sealed containers with a sterile gel media that is sterilised and set into the container before use. The container is usually made of glass or polycarbonate with a polypropylene screw lid and recycled or in a disposable polypropylene container and clip on lid. The disadvantages of this design are that you cannot change the media or treat the plants without moving to another container with an associated high labour and time cost.

Gelling agents can affect the growth of plants, but most plants which are constantly submerged in liquid media (even partially) often develop physiological conditions such as vitrification (hyperhydricity) that reduces the ability to grow or “deflask” the plants successfully. Deflasking is the process in which seedlings and clones of plants of interest that have been produced and raised in the environmental safety and luxury of a sterile PTC container are removed from the container and “introduced” to standard plant nursery conditions.

Temporary Immersion (TI) systems have successfully overcome the downsides of gelling agents and constant liquid exposure by introducing the liquid media into the plant chamber for a few minutes several times a day to allow the plants to get nutrients and exposure to phytohormones and then be drained and be exposed to lower humidity and air drying so that they don't develop any physiological issues. Most TI systems use air pressure and a complex two chamber container or a container with many internal parts to force the liquid media from the bottom up into the plant chamber, thus requiring air pumps and controls as well as air filters and strong seals to maintain a sterile system.

It is an object of the present invention to overcome or substantially ameliorate one or more of the disadvantages of prior art, or at least to provide a useful alternative.

### SUMMARY OF THE INVENTION

A first aspect of the invention provides a system for distributing a growth media to plants, comprising:
a container for receiving the plants;
a first conduit for fluidly connecting the container to a reservoir having the growth media; and
a suction device fluidly connected to the container;
wherein the suction device is operable to draw air from the container, causing the growth media to flow from the reservoir into the container and at least partially immerse the plants.

In one embodiment, the suction device operates until the growth media is substantially removed from the reservoir.

In another embodiment, after the suction device ceases to operate, the growth media is allowed to flow back into the reservoir. In a further embodiment, the pressure within the container is permitted to equalise with the pressure outside of the container to promote the flow of the growth media back to the reservoir from the container.

In some embodiments, a second conduit fluidly connects the container to the suction device.

In some embodiments, the suction device is located at, near or adjacent to a top of the container. In other embodiments, the suction device is located at, near or adjacent a side or sidewall of the container, preferably an upper side or sidewall. In further embodiments, the suction device is located at, near or adjacent to a bottom of the container. In embodiments where the second conduit fluidly connects the container to the suction device, the suction device may also be located above the container. In one embodiment, the suction device comprises a vacuum pump.

In some embodiments, the reservoir is located below the container to promote the flow of the growth media back to the reservoir from the container. In other embodiments, the reservoir is located at, near or adjacent to a bottom of the container to promote the flow of the growth media back to the reservoir from the container.

In some embodiments, the container comprises a port to permit a gas from outside the container to flow into the container. In one embodiment, the port is fluidly connected to a gas source. In another embodiment, the port is fluidly connected to air outside the container.

In some embodiments, the port is associated with a filter to remove contaminants from the gas or air. In other embodiments, the filter sterilises the gas or air.

In some embodiments, the port is configured to permit only gas or air flow in one direction. In other embodiments, the port comprises a one way valve. In further embodiments, the port comprises a two way valve.

In some embodiments, the suction device is in operation for a first predetermined period. In other embodiments, the suction device does not operate for a second predetermined period. In further embodiments, the first determined period is less than the second predetermined period.

In some embodiments, the reservoir is flexible. In one embodiment, the reservoir comprises a flexible bag.

A second aspect of the invention provides a method for distributing a growth media to plants in a container, the method comprising the steps of:
fluidly connecting the container to a reservoir having the growth media; and
fluidly connecting the container to a suction device; and
applying a suction force to draw air from the container, causing the growth media to flow from the reservoir into the container such that the plants are at least partially immersed in the growth media.

In some embodiments, the suction force is applied until the growth media is substantially removed from the reservoir.

In some embodiments, the method comprises ceasing the suction force and allowing the growth media to flow back into the reservoir. In other embodiments, the method comprises equalising the pressure within the container with the pressure outside of the container to promote the flow of the growth media back to the reservoir from the container.

In some embodiments, the suction force is applied for a first predetermined period. In other embodiments, the suction force ceases for a second predetermined period. In further embodiment, the first determined period is less than the second predetermined period.

In some embodiments, the steps of applying the suction force and ceasing the suction force are repeated to introduce the growth media into and withdraw the growth media from the container in a cycle. In other embodiments, these steps are performed continuously. In further embodiments, these steps are performed intermittently.

In some embodiments, the method comprises applying the suction force at, near or adjacent to a top of the container. In other embodiments, the method comprises applying the suction force at, near or adjacent to a side or sidewall of the container, preferably an upper side or sidewall.

In some embodiments, the method comprises locating the reservoir below the container to promote the flow of the growth media back to the reservoir from the container. In other embodiments, the method comprises locating the reservoir at, near or adjacent to a bottom of the container.

In some embodiments, the method comprises introducing a gas into the container. In one embodiment, the gas is introduced from a port fluidly connected to a gas source. In another embodiment, the gas is introduced from a port fluidly connected to air outside the container.

In some embodiments, the method comprises filtering the gas or air to remove contaminants. In other embodiments, the method comprises sterilising the gas or air.

The second aspect may have the same embodiments as the embodiments of the first aspect of the invention stated above, where applicable.

A third aspect of the invention provides a system for distributing a growth media to plants comprising:
a container for receiving the plants; and
a first conduit for fluidly connecting the container to a reservoir having the growth media;
wherein the reservoir is moveable between a dispensing position and a storage position, where the reservoir in the dispensing position allows the growth media to flow from the reservoir into the container and at least partially immerse the plants and the reservoir in the storage position allows the growth media to flow from the container into the reservoir.

In some embodiments, the dispensing position is located above the container. In other embodiments, the dispensing position is located at, near or adjacent to a top of the container. In further embodiments, the dispensing position is located at, near or adjacent to a side or sidewall of the container, preferably an upper side or sidewall.

In some embodiments, the storage position is located below the container to promote the flow of the growth media back to the reservoir from the container. In other embodiments, the storage position is located at, near or adjacent to a bottom of the container.

The third aspect may have the same embodiments as the embodiments of the first and second aspects of the invention stated above, where applicable.

A fourth aspect of the invention provides a method for distributing a growth media to plants in a container, the method comprising the steps of:
fluidly connecting the container to a reservoir having the growth media; and
moving the reservoir relative to the container to a dispensing position where the reservoir allows the growth media to flow from the reservoir into the container and at least partially immerse the plants; and
moving the reservoir relative to the container to a storage position where the reservoir allows the growth media to flow from the container into the reservoir.

The fourth aspect may have the same embodiments as the embodiments of the third aspect of the invention stated above, where applicable.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Furthermore, as used herein and unless otherwise specified, the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### BRIEF DESCRIPTION OF THE DRAWINGS

Preferred embodiments of the invention will now be described, by way of example only, with reference to the accompanying drawings in which:

FIG. 1 is a schematic side view of a system according to an embodiment of the invention in an initial state;

FIG. 2 is a schematic side view of a system according to the embodiment of FIG. 1 in an operative state;

FIG. 3 is a schematic side view of a system according to the embodiment of FIG. 1 in an inoperative state and

FIG. 4 is a schematic side view of a system according to another embodiment of the invention.

### PREFERRED EMBODIMENTS OF THE INVENTION

The present invention will now be described with reference to the following examples which should be considered in all respects as illustrative and non-restrictive. In the Figures, corresponding features within the same embodiment or common to different embodiments have been given the same reference numerals.

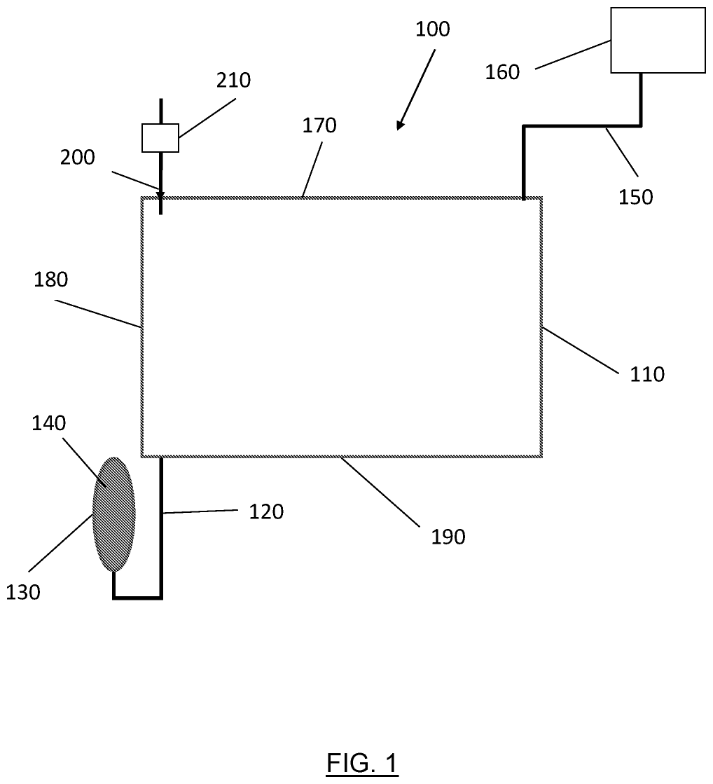

Referring to FIG. 1, a system 100 for growing plants according to a preferred embodiment of the invention comprises a container 110 for receiving the plants, a first conduit 120, a reservoir 130 having a growth media 140, a second conduit 150 and a suction device 160. The first conduit 120 fluidly connects the container 110 to the reservoir 130. The second conduit 150 fluidly connects the container 110 to the suction device 160. The suction device 160 is operable to draw air from the container 110 into the second conduit 150, causing the growth media 140 to flow from the reservoir 130 into the container and at least partially immerse the plants.

In this embodiment, the container 110 takes the form of a bioreactor substantially sealed from the exterior environment, and the first and second conduits 120, 150 take the form of flexible tubes. In other embodiments, the first and second conduits 120, 150 may take other forms, such as pipes, hoses and the like.

The suction device 160 in this embodiment is in the form of a vacuum pump located above the bioreactor 110. Alternatively, the suction device 160 can be located at, near or adjacent a top 170 of the bioreactor 110. It will be appreciated that the suction device 160 can also be located elsewhere on or near the bioreactor 110, such as a sidewall 180, preferably an upper part of the sidewall, or even a bottom 190 of the bioreactor. In another example, the suction device 160 can be located at, near or adjacent to a side of the bioreactor 110, preferably an upper side. Similarly, the suction device 160 may be located at, near or adjacent the bottom 190. It is preferred that the suction device 160 is located at least at, near or adjacent the bioreactor 110 and the second flexible tube 150 to ensure that it generates suction in the bioreactor most effectively and efficiently.

The second conduit or flexible tube 150 is located at, near or adjacent to the top 170, but can be located in the same position as or similar position to the suction device 160. However, it is preferred that the second flexible tube 150 is located so that its opening is at least at, near or adjacent the top 170 to ensure that it is not obstructed by plants or other material, like loose soil, rocks or fallen leaves, when drawing air from the bioreactor 110.

In some embodiments, the second tube 150 can be omitted and the suction device 160 may be connected directly to the bioreactor 110. In this case, the suction device 160 is directly connected to the bioreactor 110 at, near or adjacent the top 170 or the sidewall 180, preferably an upper part of the sidewall 180.

The growth media 140 is a liquid or fluid media containing nutrients to enhance plant growth and development. In FIG. 1, for clarity purposes only, the plants and associated soil are not shown but they are generally located towards the bottom 190 of the bioreactor 110.

The reservoir 130 may take the form of a flexible container and in this embodiment is a flexible bag. Preferably, the flexible bag 130 is located below the bioreactor 110, but can be placed at, near or adjacent the bottom 190.

There is also provided a port 200 located adjacent to the top 170 of the bioreactor 110 for permitting gas or (ambient) air from outside the bioreactor to flow into the bioreactor. In this embodiment, the port 200 is fluidly connected to the ambient environment external to the bioreactor 110. In this embodiment, the port comprises a two-way valve permitting air/gas to flow in either direction. However, in practice, the air/gas will flow in one direction; into the bioreactor 110 from the outside and then exit the bioreactor via the exhaust of the vacuum pump 160. However, it will be appreciated that in other embodiments, the port 200 is configured to only permit fluid to flow one way, into the bioreactor 110, and in one embodiment comprises a one-way valve. This ensures that gas/air does not escape through the port 200 during operation of the vacuum pump 160.

A filter 210 is operatively associated with the port 200 to remove contaminants from the gas or ambient air before they enter the bioreactor 110. In other embodiments, the filter 210 may also sterilise the gas or ambient air as they pass through into the bioreactor 110.

In the initial state shown in FIG. 1, the bioreactor 110 does not contain any of the growth media 140, which is entirely stored within the flexible bag 130. In addition, the air pressure inside and outside the bioreactor 110 is equal.

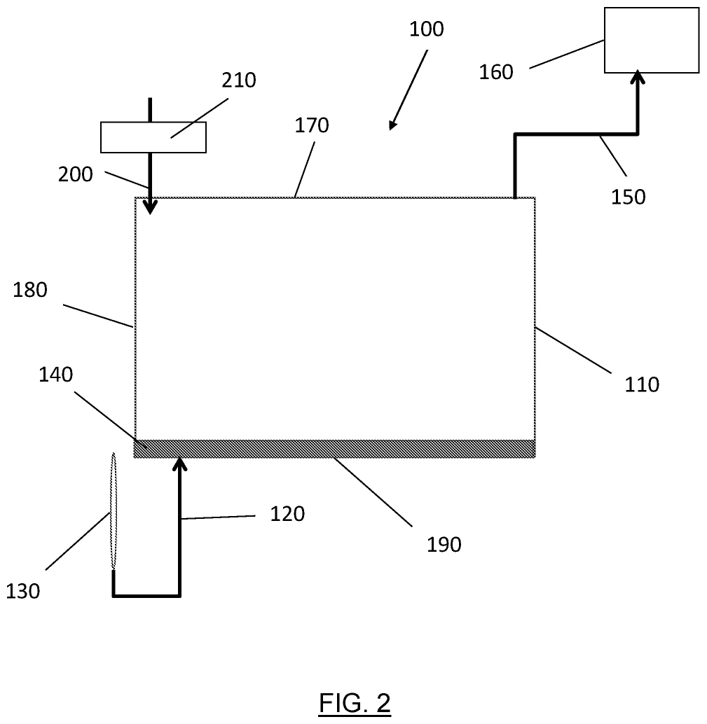

Referring to FIG. 2, when the growth media 140 is to be transported or transferred from the flexible bag 130 to the bioreactor 110, the vacuum pump 160 is activated. This causes air within the bioreactor 110 to be drawn out or flow through the flexible tube 150 to the vacuum pump 160, where it is exhausted to the ambient environment outside the bioreactor 110. This results in the air pressure inside the bioreactor 110 becoming lower than the air pressure outside the bioreactor 110 so that the liquid growth media 140 from the flexible bag 130 is drawn into the flexible tube 120 and flows through into the bioreactor 110 until the growth media is substantially removed and the flexible bag 130 has fully collapsed or deflated.

External or ambient air is also drawn in through the port 200 and the filter 210 to avoid over decompression of the bioreactor 110. The filter 210 in this embodiment is a 0.22 micron filter, which removes contaminants and thus sterilises the ambient air. However, in other embodiments, filters of various sizes may be used if sterilisation is not required but only coarse filtering of contaminants is necessary. The ingress of ambient air also enriches the air within the bioreactor 110 with fresh air. Alternatively, the port 200 can be fluidly connected to a gas source (not shown) to supplement or supply the bioreactor 110 with a gas to promote plant growth or development. For example, the port 200 can be fluidly connected to a CO₂ tank or other gas reservoir to supply CO₂ enriched gas or other gas mix to the bioreactor 110.

Once the growth media 140 is substantially removed from the flexible bag 130, the vacuum pump 160 is turned off. Alternatively, the vacuum pump 160 ceases operation when the growth media 140 has been substantially distributed within the bioreactor 110 to at least partially or fully immerse the plants located at or near the bottom 190 for a sufficient time. In a further alternative, the vacuum pump 160 reduces its suction force gradually to optimise the residence time of the growth media 140 with the plants before turning off.

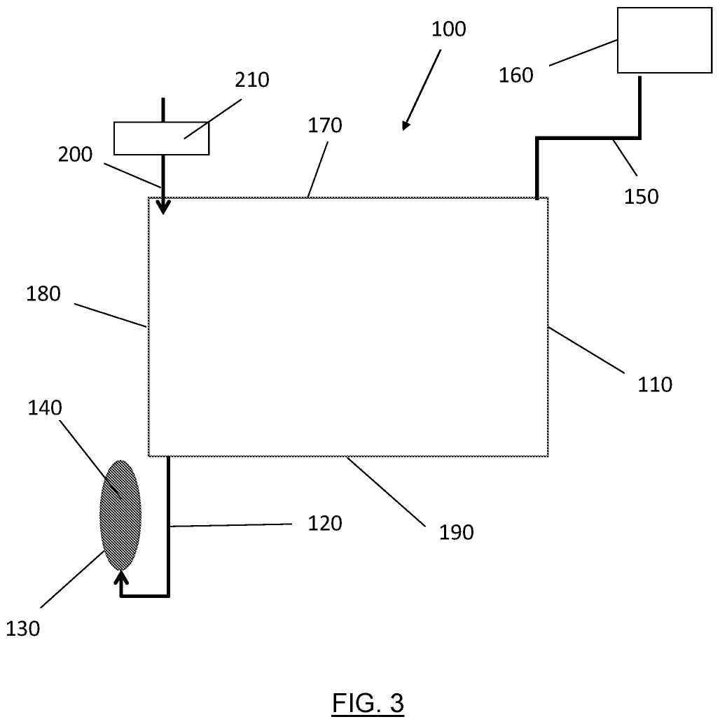

As best shown in FIG. 3, after the vacuum pump 160 ceases to operate, the growth media 140 is allowed to gradually flow back into the flexible bag 130 via the flexible tube 120. As the vacuum pump 160 is no longer generating a suction force, the pressure within the bioreactor 110 is permitted to gradually rise until it equalises with the pressure outside of the bioreactor 110. This gradual rise in pressure is facilitated by the port 200 allowing ambient air to enter the bioreactor 110. Consequently, the increased pressure promotes the flow of the growth media 140 back to the flexible bag 130 from the bioreactor 110.

In addition, gravity causes the liquid growth media 140 to drain back into the flexible bag 130, due to the location of the flexible bag 130 below the bioreactor 110. However, it will be appreciated that this effect can be similarly achieved by placing the flexible bag 130 at, near or adjacent the bottom 190 of the bioreactor 110.

This cycle of applying suction to draw the growth media 140 into the bioreactor 110 to partially immerse the plants and then ceasing suction to return the growth media to the flexible bag 130 may be repeated continuously or intermittently. Thus, the system 100 may be used to promote the growth of plants in the bioreactor 110 throughout the plant life cycle until the plants are ready for removal.

Generally, the suction force is applied for a first or operative predetermined time period to draw the growth media 140 into the bioreactor 110 and at least partially immerse the plants. This first or operative predetermined time period will usually be less than a second or inoperative predetermined time period in which there is no suction force and the growth media 140 can drain back into the flexible bag 130 for storage and/or or the growth media is not required to be dispensed to immerse the plants in the bioreactor 110.

The system 100 enables the liquid growth media 140 to be periodically introduced into the bioreactor 110 to promote the growth of the plants therein, without constantly submerging them and so avoid the development of physiological conditions such as vitrification (hyperhydricity) that reduces the ability to grow or deflask the plants. Moreover, the system 100 provides a convenient and simpler mechanism for adding and removing the growth media 140 to and from the bioreactor 110, unlike the prior art which use complex systems using complicated containers with convoluted structures, air pumps, controls and filters.

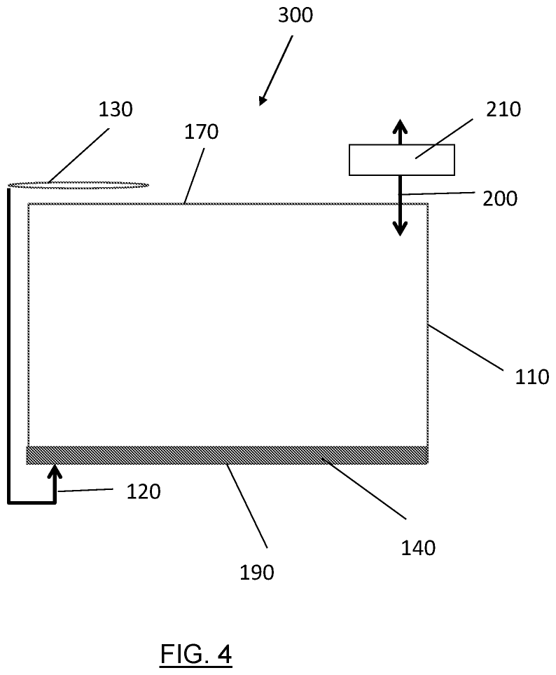

Referring to FIG. 4, another embodiment of the invention is illustrated, where the system 300 is manually operated where the vacuum pump 160 is not present or unable to be used. In this embodiment, the flexible bag 130 is placed above the bioreactor 110 so that the growth media 140 flows down through the flexible tube 120 into the bioreactor by gravity alone. As a result, there is an increase in air pressure within the bioreactor 110, which causes air to be expelled from the bioreactor 110 through the port 200 via the filter 210.

Once the growth media 140 has been distributed to the plants in the bioreactor 110 and allowed to dwell for a suitable time, the flexible bag 130 is moved or repositioned to below the bioreactor 110 so that the growth media 140 flows out of the bioreactor and into the flexible bag under the force of gravity via the flexible tube 120. At the same time, air is drawn back into the bioreactor 110 through the port 200 and filter 210 to equalise the air pressure within the bioreactor 110 with the ambient air pressure outside the bioreactor.

Accordingly, this embodiment is able to at least partially immerse plants in the bioreactor 110 with the liquid growth media 140 in a similar way to the embodiment of FIGS. 1 to 3. While this system 300 may achieve similar effects as the system 110, it does require manual intervention and hence increases labour costs. As such, the embodiment of FIGS. 1 to 3 is a more preferred way of implementing the invention in commercial applications.

It will further be appreciated that any of the features in the preferred embodiments of the invention can be combined together and are not necessarily applied in isolation from each other. For example, the reservoir or flexible bag 130 in FIGS. 1 to 3 may also be moveable as in FIG. 4 to enable both automated and manual operation of the system 100. Similar combinations of two or more features from the above described embodiments or preferred forms of the invention can be readily made by one skilled in the art.

By providing a suction device and reservoir or making the reservoir moveable relative to the container, the invention confers the advantages of being able to deliver and return growth media to a plant growing container that is convenient, simple and easy to use, especially when compared to prior art TI systems. In one embodiment, this advantage is further enhanced by providing a port to allow the passage of gas or air and a filter for controlling the air pressure within the container. All these advantages of the invention result in a TI system that uses less parts and is simpler in structure, leading to lower capital costs, less maintenance costs and greater convenience. In all these respects, the invention represents a practical and commercially significant improvement over the prior art.

Although the invention has been described with reference to specific examples, it will be appreciated by those skilled in the art that the invention may be embodied in many other forms.

1. A system for distributing a growth media to plants, comprising:
a container for receiving the plants;
a first conduit for fluidly connecting the container to a reservoir having the growth media; and
a suction device fluidly connected to the container;
wherein the suction device is operable to draw air from the container, causing the growth media to flow from the reservoir into the container and at least partially immerse the plants.

2. The system of claim 1, wherein the suction device operates until the growth media is substantially removed from the reservoir.

3. The system of claim 1 or 2, wherein, after the suction device ceases to operate, the growth media is allowed to flow back into the reservoir.

4. The system of claim 3, wherein the pressure within the container is permitted to equalise with the pressure outside of the container to promote the flow of the growth media back to the reservoir from the container.

5. The system of any one of the preceding claims, wherein a second conduit fluidly connects the container to the suction device.

6. The system of any one of the preceding claims, wherein the suction device is located at, near or adjacent to a top, side or bottom of the container.

7. The system of any one of the preceding claims, wherein the reservoir is located at, near or adjacent to a bottom of the container to promote the flow of the growth media back to the reservoir from the container.

8. The system of any one of the preceding claims, wherein the container comprises a port to permit a gas from outside the container to flow into the container.

9. The system of claim 8, wherein the port is fluidly connected to a gas source.

10. The system of claim 8, wherein the port is fluidly connected to air outside the container.

11. The system of any one of claims 8 to 10, wherein the port is associated with a filter to remove contaminants from the gas or air.

12. The system of claim 11, wherein the filter sterilises the gas or air entering the container.

13. The system of any one of the preceding claims, wherein the reservoir is flexible.

14. A method for distributing a growth media to plants in a container, the method comprising the steps of:
fluidly connecting the container to a reservoir having the growth media; and
fluidly connecting the container to a suction device; and
applying a suction force to draw air from the container, causing the growth media to flow from the reservoir into the container such that the plants are at least partially immersed in the growth media.

15. The method of claim 14, wherein the suction force is applied until the growth media is substantially removed from the reservoir.

16. The method of claim 14 or 15, comprising ceasing the suction force and allowing the growth media to flow back into the reservoir.

17. The method of claim 15 or 16, wherein the steps of applying the suction force and ceasing the suction force are repeated to introduce the growth media into and withdraw the growth media from the container continuously or intermittently.

18. The method of any one of claims 14 to 17, comprising equalising the pressure within the container with the pressure outside of the container to promote the flow of the growth media back to the reservoir from the container.

19. The method of any one of claims 14 to 18, comprising locating the reservoir at, near or adjacent to a bottom of the container to promote the flow of the growth media back to the reservoir from the container.

20. The method of any one of claims 14 to 19, comprising introducing a gas into the container.

21. The method of claim 20, wherein the gas is introduced from a port fluidly connected to a gas source.

22. The method of claim 20, wherein the gas is introduced from a port fluidly connected to air outside the container.

23. The method of any one of claims 20 to 22, comprising filtering the gas or air to remove contaminants.

24. The method of any one of claims 20 to 22, comprising sterilising the gas or air.

25. The method of any one of claims 14 to 24, comprising applying the suction force at, near or adjacent to a top of the container.
